(19) United States
(12) Patent Application Publication
(10) Pub. No.: US 2008/0069879 A1
(43) Pub. Date: Mar. 20, 2008

(54) STABLE SOLID DOSAGE FORM CONTAINING AMORPHOUS CEFDITOREN PIVOXIL AND PROCESS FOR PREPARATION THEREOF

(76) Inventors: Ravishekhar Bhiwgade, Wardha (IN); Romi Barat Singh, Uttar Pradesh (IN)

Correspondence Address: Jayadeep R. Deshmukh;Ranbaxy Inc., Suite 2100, 600 College Road East, Princeton, NJ, 08540, US

(21) Appl. No.: 11/742,799

(22) Filed: May 1, 2007

(30) Foreign Application Priority Data
May 2, 2006 (IN) 1106/DEL/2006

(51) Int. Cl. A61K 31/545; A61K 9/24

(52) U.S. Cl. 424/472; 427/2.14; 514/202

(57) ABSTRACT

The present invention relates to stable solid dosage form and a dry process for preparing amorphous cefditoren pivoxil solid dosage forms and coating the solid dosage form with one or more layers of aqueous dispersion of film forming agents.

### FIELD OF THE INVENTION

The present invention relates to stable solid dosage form and a dry process for preparing amorphous cefditoren pivoxil solid dosage forms and coating the solid dosage form with one or more layers of aqueous dispersion of film forming agents.

### BACKGROUND OF THE INVENTION

‘Cefditoren’ is the generic name of cepham compound: 7-[2-methoxyimino-2-(2-aminothiazol-4-yl)acetamido]-3-[2-(4-methylthiazol-5-yl)vinyl]-3-cephem-4-carboxylic acid (syn-isomer, cis-isomer). Cefditoren synthesis is disclosed in U.S. Pat. Nos. 4,839,350 and 4,918,068. An injectable cefditoren preparation is disclosed in U.S. Pat. No. 5,595,986. Cefditoren pivoxil, synthesized by forming a pivaloyloxymethyl (pivoxil)ester with cefditoren at the carboxylic acid moiety, exhibits improved oral absorption and is quickly hydrolyzed to cefditoren by enzymatic esterases upon absorption.

Cefditoren exhibits a broad antibacterial spectrum relatively having low toxicity and is useful in the treatment or prophylaxis of diseases induced by gram-positive and gram-negative bacteria. Cefditoren pivoxil is, by itself, antibacterially inactive but is useful as a prodrug. Cefditoren pivoxil is administrable orally and can be converted into the antibacterially active cefditoren in the digestive tract of mammals, with cleaving of the ester-forming pivaloyloxymethyl group therefrom.

Crystalline cefditoren pivoxil is known to have high purity, high heat stability and in addition satisfactory stability even when stored under high humidity conditions (U.S. Pat. No. 6,294,669). However, crystalline cefditoren pivoxil has low solubility in water and thus is not suitable for oral administration. Low-solubility drugs often show poor bioavailability or irregular absorption, the degree of irregularity being affected by factors, such as dose level, fed state of the patient, and form of the drug.

Over the years, compositions and methods were developed to achieve improved solubility of such poorly or sparingly soluble drugs.

U.S. Patent Application No. 2003/0060451 discloses a method of preventing premature de-esterification of pro-drug esters and enhancing the oral bioavailability by formulating the prodrug ester i.e., cefditoren pivoxil in a non-emulsified formulation with lecithin.

One of the reported methods involves the conversion of a medicinal compound that is sparingly soluble in water into an amorphous substance, thus to improve the solubility of the compound in water. Conversion of crystalline cefditoren pivoxil to an amorphous form leads to high water solubility and improves the usefulness of cefditoren pivoxil in the therapy of disease.

U.S. Pat. Nos. 6,342,493 and 6,486,149 disclose the conversion of crystalline cefditoren pivoxil to amorphous form. In such conversion, crystalline cefditoren pivoxil is dissolved in an acidic aqueous solution containing a water-soluble polymeric additive, the acidic aqueous solution neutralized to coprecipitate cefditoren pivoxil together with the water-soluble polymeric additive, and the precipitate is then collected, washed and dried. According to this method, a yellow-colored powdery composition comprising solid particles of an intimate mixture of an amorphous form of cefditoren pivoxil having a high level of dissolvability in water and high heat stability with the water-soluble polymeric additive (0.5 to 5%) can be provided. This method, however, involves many steps and thus requires process control and is time consuming.

U.S. Patent Application No 2004/0115272 discloses a method of conversion of crystalline cefditoren pivoxil to amorphous form by grinding crystalline cefditoren pivoxil in the presence of a pharmaceutically acceptable organic polymeric compound.

EP 0629404 discloses a pharmaceutical composition comprising cefditoren pivoxil and a water soluble polymer-like hydroxypropylcellulose. This preparation was disclosed to have improved wettability, dispersibility and absorbability without increasing its bitterness. Improved dispersibility and absorbability was also disclosed for a composition containing cefditoren pivoxil and β-cyclodextrin along with an ionic surfactant in a pharmaceutically acceptable carrier (EP 0339465).

U.S. Patent Application No. 2006/0051411 discloses a pharmaceutical composition comprising amorphous cefditoren pivoxil and a sugar ester fatty acid, which was obtained by mixing or wet-granulating particles containing amorphous cefditoren pivoxil with the sugar ester fatty acid while amorphous cefditoren pivoxil maintains its particle state.

Amorphous formulations with improved bioavailability have a disadvantage: amorphous materials are thermodynamically unstable and therefore show some tendency to crystallize spontaneously. The transition from amorphous to crystalline form depends on the molecular mobility, which is related to the glass transition temperature (T_{g}). Many amorphous materials are plasticized by sorbed water. Once incorporated into the amorphous region, water can increase the free volume and lead to enhance molecular mobility within the amorphous material, causing crystallization.

In addition, it is found that most of amorphous materials are very fine and fluffy material, with relatively low bulk and tap density. This property can make it difficult to formulate into a dosage form with uniformity of weight, hardness and other desirable tablet properties. Wet granulation can solve this problem, but it should to be avoided, as addition of a solvent along with, and subsequent removal in way of drying the granules at elevated temperatures may covert the amorphous form to crystalline form. Direct compression could be a method of choice for preparing the formulations containing amorphous materials.

WO 2005/087198 discloses a preparation of a pharmaceutical solid dosage form comprising amorphous drug material by dry process. The formulation was disclosed to have improved stability even after storage for two months at 40° C. and 75% RH.

Cefditoren pivoxil, apart from exhibiting low solubility, has another unfavorable property—bitterness. Cefditoren does not exhibit the bitter taste by itself upon oral administration thereof, whilst cefditoren pivoxil exhibits a strong bitter taste on oral administration. Thus, there is a need that the bitter taste of cefditoren pivoxil should be minimized to such an extent that the oral administration of cefditoren pivoxil would be palatable to patients.

U.S. Patent Application No. 2003/0026843 discloses a method relating to amorphous drug beads comprising an amorphous active drug and an organic surfactant (casein) having improved solubility, absorption and wettability characteristics.

Addition of a water-soluble casein salt to cefditoren pivoxil has been disclosed in U.S. Pat. No. 5,958,915 as a method for enhancing solubility of the drug, with minimized bitter taste. However, this method includes the steps of granulating, drying, milling, compressing, etc, for preparing the tablets, which is time consuming and tedious.

In addition, consumption of formulations using water-soluble casein may cause difficulties for individuals with lactose intolerance because of their inability to digest significant amounts of lactose, which is the predominant sugar. In such conditions, use of synthetic surfactant can be an alternative way in administering such poorly water-soluble drug products.

Various methods for masking or reducing the bitter taste of medicinal compositions or formulations containing a drug compound having a bitter taste are generally known. For instance, known methods of masking the bitter taste of a drug compound embraces a method of coating the surfaces of the particles of the drug compound with a film coating. This method of coating particle surfaces can suitably be applied to the preparations of the tablet form.

Film coating of pharmaceuticals is a common manufacturing stage for the following reasons: (i) to provide physical and chemical protection for the drug, (ii) to mask the taste, odor or color of the drug or (iii) to control the release rate or site of the drug from the tablet. When a coating composition is applied to a batch of tablets or granules, the core surfaces become covered with a polymeric film that is formed as the surfaces dries. The major component in a coating formulation is a film forming agent, which ideally is a high molecular-weight polymer that is soluble or dispersible in the proper solvent (today, most preferably in aqueous-based media). The polymer forms a gel and produces an elastic, cohesive and adhesive film coating.

In the pharmaceutical industry, organic solvent-based film coatings have been used for over 40 years. However, interest in the use of aqueous-based film coating systems has been rapidly increasing owing to the well-documented drawbacks (unsafe, toxic, polluting and uneconomic) associated with organic-solvent-based coating systems. Consequently, and for the reasons mentioned above, efforts have been focused on identifying new aqueous-based film coating formulations. To date, aqueous-soluble/dispersible polymers available on the market primarily include either cellulosic polymers or acrylate copolymers, such as hydroxypropyl methylcellulose (HPMC), cellulose acetate phthalate (CAP), hydroxypropyl methylcellulose acetate succinate (HPMCAS), and methacrylic acid polymers and copolymers. An example of an aqueous dispersion for conventional film coating of tablets is Opadry® (HPMC). There are, however, some material related limitations in using these polymers in aqueous-based film coating, such as logo-bridging, cracking and edge splitting.

WO 2002/092708 discloses a method for preparing an aqueous coating dispersion of starch having good film-forming properties and usability in the room temperature. The films have satisfactory mechanical strength properties and as a consequence of their hydrophilicity good oxygen barriers.

However, no known methods provide an acceptable dosage form containing cefditoren pivoxil with improved solubility and stability employing a simple process which is easy, cost-effective and time saving.

Thus, there is a need for a simple method of producing a cefditoren pivoxil dosage form with enhanced stability, which does not require wet granulation with organic solvents or water and do not require an additional step of drying.

### SUMMARY OF THE INVENTION

In one aspect, provided are processes for preparing a pharmaceutical composition comprising the steps of providing a solid dosage form comprising amorphous cefditoren pivoxil, and coating the solid dosage form with one or more layers of aqueous dispersion of film forming agents.

The processes may include one or more of the following embodiments. For example, amorphous cefditoren pivoxil can constitute about 20 to 80% (w/w) based on the total weight of the pharmaceutical composition. In another embodiment, the solid dosage form can be provided by a dry process comprising dry granulation or direct compression of amorphous cefditoren pivoxil. Other suitable dry processes may be used.

In yet another embodiment, the aqueous dispersion comprising one or more film forming agents can constitute about 1 to 10% (w/w) based on the total weight of the pharmaceutical composition and the film forming agents can be selected from one or more of hydroxypropyl methylcellulose, hydroxypropyl cellulose, methyl cellulose, carboxymethylcellulose, hydroxymethyl cellulose, hydroxyethylcellulose, ethyl cellulose, hydroxypropyl methyl phthalate, cellulose acetate, cellulose acetate trimelliatate, cellulose acetate phthalate, waxes, copolymers of acrylic and methacrylic acid or mixtures thereof.

In another embodiment, the dosage can form further comprise one or more surfactants. Surfactants can constitute about 1 to 20% (w/w) based on the total weight of the pharmaceutical composition and can comprise at least one of anionic, cationic, zwitterionic or nonionic surfactants. Particular surfactants include one or more anionic surfactants and more particularly, the surfactant can be sodium lauryl sulfate.

In another embodiment, the solid dosage form can further comprise one or more pharmaceutically acceptable excipients. Suitable pharmaceutically acceptable excipients can comprise at least one or more fillers, binders, disintegrants, lubricants, glidants, coloring agents, flavoring agents or mixtures thereof.

In another embodiment, providing the solid dosage form can comprise the steps of:
a. blending amorphous cefditoren pivoxil and one or more pharmaceutically acceptable excipients to form a blend,
b. compacting or slugging the blend to form compacts,
c. milling and sizing the compacts to form granules,
d. mixing the granules with one or more of pharmaceutically acceptable excipients and compressing to form a tablet, and
e. coating the tablet with one or more layers of aqueous dispersion comprising one or more film forming agents.

In yet another embodiment, providing the solid dosage form can comprise the steps of:
a. blending amorphous cefditoren pivoxil, a surfactant, and one or more pharmaceutically acceptable excipients to form a blend,
b. compacting or slugging the blend to form compacts,
c. milling and sizing the compacts to form granules,
d. mixing the granules with one or more of pharmaceutically acceptable excipients and compressing to form a tablet, and
e. coating the tablet with one or more layers of aqueous dispersion comprising one or more film forming agents.

In yet another embodiment, providing the solid dosage form can comprise the steps of:
a. blending amorphous cefditoren pivoxil and one or more pharmaceutically acceptable excipients to form a blend,
b. directly compressing the blend formed in step (a) into a tablet, and
c. coating the tablet with one or more layers of aqueous dispersion comprising one or more film forming agents.

In yet another embodiment, providing the solid dosage fonts can comprise the steps of:
a. blending amorphous cefditoren pivoxil, a surfactant, and one or more pharmaceutically acceptable excipients to form a blend,
b. directly compressing the blend formed in step (a) into a tablet and
c. coating the tablets with one or more layers of aqueous dispersion of film forming agents.

In another embodiment, the solid dosage form can further comprise one or more additional antibacterial agents.

In another aspect, provided are pharmaceutical compositions, which comprises a solid dosage form comprising amorphous cefditoren pivoxil, optionally one or more pharmaceutically acceptable excipients and optionally one or more surfactants, wherein the solid dosage form is prepared by a dry process and is coated with one or more layers of aqueous dispersion comprising one or more film forming agents.

The pharmaceutical composition can comprise one or more of the following embodiments. For example, the dry process can be selected from dry granulation or direct compression.

In another embodiment, the one or more film forming agents can be selected from one or more of hydroxypropyl methylcellulose, hydroxypropyl cellulose, methyl cellulose, carboxymethylcellulose, hydroxymethyl cellulose, hydroxyethylcellulose, ethyl cellulose, hydroxypropyl methyl phthalate, cellulose acetate, cellulose acetate trimelliatate, cellulose acetate phthalate, waxes, copolymers of acrylic and methacrylic acid or mixtures thereof.

In yet another embodiment, the one or more surfactants can constitute about 1 to 20% (w/w) based on the total weight of the pharmaceutical composition and can comprise at least one of anionic, cationic, zwitterionic or nonionic surfactants.

In another embodiment, the one or more pharmaceutically acceptable excipients can comprise at least one or more fillers, binders, disintegrants, lubricants, glidants, coloring agents, flavoring agents or mixtures thereof.

In another embodiment, the solid dosage form can further comprise one or more additional antibacterial agents.

In another aspect, provided are methods for treating a bacterial infection by administering to a person in need thereof a pharmaceutical composition comprising a solid dosage form, which comprises amorphous cefditoren pivoxil, optionally one or more pharmaceutically acceptable excipients and optionally one or more surfactants, wherein the solid dosage form is prepared by a dry process and is coated with one or more layers of aqueous dispersion comprising one or more film forming agents.

The method can further comprise concurrently or sequentially administering one or more additional antibacterial agents.

### DETAILED DESCRIPTION OF THE INVENTION

Provided are processes for preparing pharmaceutical compositions, which comprises a solid dosage form comprising amorphous cefditoren pivoxil, optionally one or more suitable surfactants, by a dry process. The solid dosage form can be further coated with one or more layers of aqueous dispersion comprising one or more film forming agents, to enhance its physicochemical stability.

The aqueous dispersion coating over the solid dosage forms ensures that the amorphous form of cefditoren pivoxil remains thermodynamically stable and resistant to crystallization. Generally, amorphous materials are plasticized by sorbed water, leading to increase in the free volume and enhanced molecular mobility, causing crystallization. Aqueous dispersion coating over the solid dosage forms of cefditoren pivoxil reduces the molecular mobility in significant way and has good stability during storage, over other solvent coatings.

In addition, the processes provided herein are simple, economical and environmentally friendly, as it does not require any solvents that are commonly used in conventional coating processes.

Examples of suitable film forming agents include, but are not limited to, hydroxypropyl methylcellulose, hydroxypropyl cellulose, methyl cellulose, carboxymethylcellulose, hydroxymethylcellulose, hydroxyethylcellulose, ethyl cellulose, hydroxypropyl methyl phthalate, cellulose acetate, cellulose acetate trimelliatate, cellulose acetate phthalate; waxes, such as polyethylene glycol; copolymers of acrylic and methacrylic acid, such as those marketed under the brand names Eudragit® RL and RS; or mixtures thereof. Alternatively, commercially available coating compositions comprising film-forming polymers marketed under various trade names, such as Opadry®, may also be used for coating.

The aqueous dispersion coating can be present from about 1 to 10% (w/w) based on the total weight of the pharmaceutical composition.

Coatings may be applied as solutions/dispersions of coating ingredients using any conventional technique known in the prior art, such as spray coating in a conventional coating pan or fluidized bed processor or dip coating.

Processes for preparing solid dosage forms comprising amorphous cefditoren pivoxil, optionally a suitable surfactant, can include dry processes such as, dry granulation and direct compression. The dry processes as such have resistance for the conversion of amorphous form to crystalline form. The dry processes include any methods known in the art for preparing solid dosage forms by dry processes, as well as by methods described in co-pending Indian Patent Application No. 445/DEL/2006, which is incorporated herein in its entirety.

Solid dosage forms may be prepared by mixing amorphous cefditoren pivoxil, optionally with one or more surfactants and one or more pharmaceutically acceptable excipients and forming into solid dosage forms. The solid dosage form can be further coated with one or more layers of aqueous dispersion comprising one or more film forming agents. Amorphous cefditoren pivoxil can be prepared by any methods known in the art, as well as by methods described in co-pending Indian Patent Application No. 207/DEL/2005, which is incorporated herein in its entirety.

Cefditoren pivoxil can be utilized in an amount of ranging from 20 to 80% (w/w) based on the total weight of the pharmaceutical composition.

Pharmaceutical compositions described herein may contain one or more surfactants. Suitable surfactants can be anionic, cationic, zwitterionic or nonionic surfactants. Preferably, the compositions include at least one anionic surfactant. Suitable anionic surfactants include, but are not limited to, alkyl sulfonates, alkyl phosphates, alkyl phosphonates, potassium laureate, sodium lauryl sulfate, sodium dodecylsulfate, alkyl polyoxyethylene sulfates, dioctyl sodium sulfosuccinate, phosphatidyl glycerol, phosphatidylinositol, diphosphatidylglycerol, phosphatidyl inosine, phosphatidylserine, phosphatidic acid and their salts, cholic acid and other bile acids (e.g., cholic acid, deoxycholic acid, glycocholic acid, taurocholic acid, glycodeoxycholic acid) and salts thereof (e.g., sodium deoxychelate, etc.).

Surfactants may be added in an amount of about 1 to 20% (w/w) based on the total weight of the pharmaceutical composition. Surfactants can help in increasing the solubility of cefditoren pivoxil and thereby can increase dissolution rate.

Pharmaceutically acceptable excipients include those known in the art and may be selected from fillers, binders, disintegrants, lubricants, glidants coloring agents, flavoring agents or mixtures thereof. Pharmaceutically acceptable excipients may be present intragranularly or extragranularly or both.

Examples of fillers include, but are not limited to, corn starch, lactose, white sugar, sucrose, sugar compressible, sugar confectioners, glucose, sorbitol, calcium carbonate, calcium phosphate-dibasic, calcium phosphate-tribasic, calcium sulfate, microcrystalline cellulose, silicified microcrystalline cellulose, cellulose powdered, dextrates, dextrins, dextrose, fructose, kaolin, lactitol, mannitol, starch, pregelatinized starch or mixtures thereof.

Examples of binders include, but are not limited to, methyl cellulose, hydroxypropyl cellulose, hydroxypropyl methylcellulose, polyvinylpyrrolidone, gelatin, gum Arabic, ethyl cellulose, polyvinyl alcohol, pregelatinized starch, agar, tragacanth, sodium alginate, propylene glycol or mixtures thereof.

Examples of disintegrants include, but are not limited to, starch, cross-linked carboxy methyl cellulose sodium, cross-linked polyvinyl pyrrolidone, sodium starch glycolate, low substituted hydroxypropyl cellulose or mixtures thereof.

Examples of lubricants and glidants include, but are not limited to, colloidal anhydrous silica, stearic acid, magnesium stearate, calcium stearate, talc, hydrogenated castor oil, sucrose esters of fatty acids, microcrystalline wax, yellow beeswax, white beeswax or mixtures thereof.

Coloring agents and flavoring agents may be selected from any FDA approved colors and flavors for oral use.

Amorphous cefditoren pivoxil solid dosage forms may be formulated into various pharmaceutical compositions suitable for oral administration, e.g., in the form of tablet or capsule in accordance with any of the conventional procedure known in the art.

In one general aspect, amorphous cefditoren pivoxil can be optionally mixed with one or more surfactant, and/or one or more of pharmaceutical excipients, such as a filler, binder, disintegrant, lubricant or mixture thereof in a suitable blender. The resultant blend can either be directly compressed into solid dosage form or compacted to granules by roller compaction. The dry process prevents the conversion of amorphous cefditoren pivoxil to crystalline form.

The compaction of the drug or the mixture comprising the drag and excipient(s) into compacts may be carried out by slugging or by roller compaction, particularly roller compaction.

Roller compactor functions by uniformly applying pressure on a mixed powder blend by passing the blend between two counter-rotating rollers. The pressure imparted on the blend by the rollers compresses the powder into a compact, such as a sheet or ribbon, which is typically milled to produce granules.

Granules obtained as above may be filled into capsules or packed in a sachet. Alternatively, granules may be mixed with one or more of pharmaceutically acceptable excipients and/or surfactants and compressed into tablets.

As an alternative to roller compaction, slugging may be used for preparing solid dosage forms, such as a tablet. The process is simple, low in cost and effective. Slugging may be carried out by means of a tablet press. The drug either alone or mixed with one or more optional surfactant and/or other excipients can be pre-compressed on a heavy duty press. The slug so formed can be milled into granules and recompressed into tablet. The granules may also optionally be mixed with other extragranular excipients prior to compression into a tablet.

Both the processes, i.e., roller compaction and slugging, generate fines during the milling step. A portion of these fines can be mixed with granules and compressed into a tablet or can be easily recycled by collecting them and compacting again.

In one embodiment, processes provided herein comprise blending amorphous cefditoren pivoxil, a surfactant, filler, binder, disintegrant and lubricant and compacting the mixture using roller compactor; milling and sizing the compacts into granules with a desired particle size distribution; mixing with one or more of filler, binder, disintegrant and lubricant and compressed into a tablet using appropriate tooling.

Alternative to dry granulation, direct compression may be carried out for preparing solid dosage forms, such as tablets. The direct compression process for preparing cefditoren pivoxil tablets comprises mixing amorphous cefditoren pivoxil, optionally a surfactant with one or more pharmaceutically acceptable excipients using conventional blending equipment, under low-shear conditions. Tire blended formulation is thereafter directly compressed using conventional types of tableting equipments.

Pharmaceutical compositions containing amorphous cefditoren pivoxil can be used as antibacterial agents. Bacteria referred to herein include for example gram-positive bacteria such as staphylococcus and streptococcus, gram-negative bacteria such as Escherchia coli, Branhamella catarrhalis, klebsiella, Proteus, and Haemophilus influenzae, and anaerobes such as Peptostreptococcus, Propionibacterium acnes, and Bactericides. Further, pharmaceutical compositions described herein can be useful for the prophylaxis or therapy of diseases induced by gram-positive bacteria or gram-negative bacteria.

Pharmaceutical compositions described herein may be administered in combination with other medicines, for example, other antibacterial agents.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

### Example 1

### Amorphous Cefditoren Pivoxil 1

| Ingredient | Quantity | |
|---|---|---|
| Cefditoren Pivoxil | 20.0 | g |
| Crospovidone | 20.0 | g |
| Propylene Glycol | 1.0 | g |
| Acetone | 2000 | mL |

Propylene glycol was dissolved in acetone. Cefditoren pivoxil was added to the solution and crospovidone was dispersed into the solution. The solvent was removed to obtain the amorphous cefditoren pivoxil.

### Example 2

### Amorphous Cefditoren Pivoxil 2

| Ingredient | Quantity | |
|---|---|---|
| Cefditoren Pivoxil | 20.0 | g |
| Colloidal silicon dioxide | 3.0 | g |
| Hydroxypropyl cellulose | 1.2 | g |
| Acetone | 2000 | mL |

Hydroxypropyl cellulose was dissolved in acetone. Cefditoren pivoxil was added to the solution and colloidal silicon dioxide was dispersed into the solution. The solvent was removed to obtain the amorphous cefditoren pivoxil.

### Example 3

### Amorphous Cefditoren Pivoxil 3

| Ingredient | Quantity | |
|---|---|---|
| Cefditoren Pivoxil | 1.0 | g |
| Colloidal silicon dioxide | 0.15 | gm |
| Acetone | 100 | mL |

Cefditoren pivoxil was dissolved in acetone and colloidal silicon dioxide was dispersed into the solution. The solvent was removed to obtain the amorphous cefditoren pivoxil.

### Example 4

### Amorphous Cefditoren Pivoxil 4

| Ingredient | Quantity | |
|---|---|---|
| Cefditoren Pivoxil | 1.0 | g |
| Colloidal silicon dioxide | 0.15 | gm |
| Hydroxypropyl methylcellulose | 0.06 | gm |
| Dichloromethane | 20.0 | mL |
| Isopropyl alcohol | 5.0 | mL |

Hydroxypropyl methylcellulose was dissolved in a mixture of dichloromethane and isopropyl alcohol to form a solution. Cefditoren pivoxil was added to the solution and colloidal silicon dioxide was dispersed into the solution. The solvent was removed to obtain the amorphous cefditoren pivoxil.

### Example 5

### Preparation of Pharmaceutical Composition

| | |
|---|---|
| Ingredients | Percent (%) w/w |

Intragranular

| | |
|---|---|
| Amorphous cefditoren pivoxil of Example 4 | 48.89 |
| Mannitol | 27.8 |
| Cross-linked carboxy methyl cellulose sodium | 7.8 |
| Sodium lauryl sulfate | 1.9 |
| Hydroxypropyl cellulose | 1.6 |
| Magnesium Stearate | 0.3 |

Extragranular

| | |
|---|---|
| Cross-liked carboxy methyl cellulose sodium | 7.8 |
| Colloidal silicon dioxide | 0.3 |
| Magnesium Stearate | 0.7 |

Coating

| | |
|---|---|
| Colorcon Opadry OYS-58910 | 2.8 |
| Solvent system - Purified Water | 10% dispersion |

### Procedure:

1. Amorphous cefditoren pivoxil was blended with mannitol, cross-linked carboxy methyl cellulose sodium, sodium lauryl sulfate, hydroxypropyl cellulose and magnesium stearate and compacted using roller compactor.
2. The compacts prepared in step 1 were sized into granules by milling and blended with extragranular cross-linked carboxy methyl cellulose sodium, colloidal silicon dioxide and magnesium stearate.
3. The blend obtained from step 2 was compressed into tablets using appropriate tooling.
4. The compressed tablets were coated with above coating composition.

### Example 6

### Preparation of Pharmaceutical Composition

| | |
|---|---|
| Ingredients | Percent (%) w/w |

Intragranular

| | |
|---|---|
| Amorphous cefditoren pivoxil (Example 4) | 47.9 |
| Mannitol | 29.1 |
| Cross-linked carboxy methyl cellulose sodium | 7.7 |
| Hydroxypropyl cellulose | 1.5 |
| Magnesium Stearate | 0.3 |

Extragranular

| | |
|---|---|
| Cross-linked carboxy methyl cellulose sodium | 7.7 |
| Colloidal silicon dioxide | 0.3 |
| Magnesium Stearate | 0.7 |

Coating

| | |
|---|---|
| Colorcon Opadry OYS-58910 | 4.8 |
| Solvent system - 3:2 mixture of Isopropyl alcohol and | 5% dispersion |
| dichloromethane. | |

### Procedure:

1. Amorphous cefditoren pivoxil was blended with mannitol, cross-linked carboxy methyl cellulose sodium, sodium lauryl sulfate, hydroxypropyl cellulose and magnesium stearate and compacted using roller compactor.
2. The compacts prepared in step 1 were sized into granules by milling and blended with extragranular cross-linked carboxy methyl cellulose sodium, colloidal silicon dioxide and magnesium stearate.
3. The blend obtained from step 2 was compressed into tablets using appropriate tooling.
4. The compressed tablets were coated with above coating composition.

### Example 7

### Stability of Pharmaceutical Compositions

Tablets prepared in Examples 5 and 6 were subjected to accelerated stability studies at 40° C. and 75% relative humidity (RH) for the period of one month and three months. The samples were analyzed for the presence of crystallinity. The results are provided in Table 1.

TABLE 1
Crystallinity detection from cefditoren pivoxil tablets subjected
to stability studies under 40° C. with 75% RH for the
period 1 month and 3 months

| Sample | Initial | After 1 month | After 3 months |
|---|---|---|---|
| Example 5 | Not Detected | Not Detected | Not Detected |
| Example 6 | Not Detected | Detected | — |

### Example 8

Tablets prepared in the Example 5 were studied for other stability parameters—water content, dissolution and related substances after charging them at accelerated stability condition of 40° C. and 75% RH in HDPE bottles for the period of three months. The results of this study are provided in Tables 2, 3 and 4.

TABLE 2
Water content detection by Karl Fischer (KF) method for cefditoren
pivoxil tablets of Example 5, subjected to stability studies under
40° C. with 75% RH for the period 3 months.

| Parameters | Initial | After 3 months |
|---|---|---|
| Water content (% w/w) by | 0.55 | 0.42 |
| KF in methanol | | |

TABLE 3
In vitro release of cefditoren pivoxil from tablets prepared according
to Example 5, subjected to stability studies under 40° C. with
75% RH for the period 3 months, in 900 mL water, USP Apparatus II,
at 75 rpm and 37° C.

Percentage (%)
of cefditoren
pivoxil released

| Time (minutes) | Initial | After 3 months |
|---|---|---|
| 5 | 99 | 92 |
| 10 | 99 | 94 |
| 15 | 99 | 95 |
| 20 | 100 | 95 |
| 25 | 100 | 94 |

TABLE 4
Related substances detection from Example 5, which was
subjected to stability studies under 40° C. with
75% RH for the period 3 months

| Related substances | initial | After 3 months |
|---|---|---|
| Delta-2 isomer of Cefditoren Acid | 0.053 | 0.032 |
| Cefditoren Acid | 0.154 | 0.157 |
| Methyl ester of Cefditoren | ND | ND |
| D-Lactam Cefditoren pivoxil | 0.437 | 0.328 |
| Ceftamate pivoxil | 0.089 | 0.113 |
| Delta-2 Cefditoren pivoxil | 1.443 | 2.024 |
| Cefditoren pivoxil E-isomer | 0.441 | 0.524 |
| Specified impurity (MW 620) | 0.037 | 0.037 |
| N-pivoloyal Cefditoren pivoxil | 0.102 | 0.119 |
| Dimer (MW 1252) | 0.871 | 1.079 |
| Specified impurity (MW 1241) | 0.585 | 0.774 |
| Dimer (MW 1367) | 0.092 | 0.091 |
| Highest Unknown | 0.006 | 0.028 |
| Total Unknown | 0.014 | 0.028 |
| Total RS | 3.594 | 4.459 |

We claim:

1. A process for preparing a pharmaceutical composition comprising the steps of: providing a solid dosage form comprising amorphous cefditoren pivoxil, and coating the solid dosage form with one or more layers of aqueous dispersion comprising one or more film forming agents,

2. The process of claim 1, wherein the amorphous cefditoren pivoxil constitutes about 20 to 80% (w/w) based on the total weight of the solid dosage form.

3. The process of claim 1, wherein the solid dosage form is provided by a dry process comprising dry granulation or direct compression of amorphous cefditoren pivoxil.

4. The process of claim 1, wherein the aqueous dispersion comprising one or more film forming agents constitutes about 1 to 10% (w/w) based on the total weight of the solid dosage form and the film forming agents are selected from one or more of hydroxypropyl methylcellulose, hydroxypropyl cellulose, methyl cellulose, carboxymethylcellulose, hydroxymethyl cellulose, hydroxyethylcellulose, ethyl cellulose, hydroxypropyl methyl phthalate, cellulose acetate, cellulose acetate trimelliatate, cellulose acetate phthalate, waxes, copolymers of acrylic and methacrylic acid or mixtures thereof.

5. The process of claim 1, wherein the solid dosage form further comprises one or more surfactants.

6. The process of claim 5, wherein the one or more surfactants constitutes about 1 to 20% (w/w) based on the total weight of the solid dosage form and comprises at least one of anionic, cationic, zwitterionic or nonionic surfactants.

7. The process of claim 1, wherein the solid dosage form further comprises one or more pharmaceutically acceptable excipients.

8. The process of claim 7, wherein the one or more pharmaceutically acceptable excipients comprise at least one or more fillers, binders, disintegrants, lubricants, glidants, coloring agents, flavoring agents or mixtures thereof.

9. The process of claim 1, wherein providing the solid dosage form comprises:
a. blending amorphous cefditoren pivoxil and one or more pharmaceutically acceptable excipients to form a blend,
b. compacting or slugging the blend to form compacts,
c. milling and sizing the compacts to form granules,
d. mixing the granules with one or more of pharmaceutically acceptable excipients and compressing to form a tablet, and
e. coating the tablet with one or more layers of aqueous dispersion comprising one or more film forming agents.

10. The process of claim 1 comprising the steps of:
a. blending amorphous cefditoren pivoxil, a surfactant, and one or more pharmaceutically acceptable excipients to form a blend,
b. compacting or slugging the blend to form compacts,
c. milling and sizing the compacts to form granules,
d. mixing the granules with one or more of pharmaceutically acceptable excipients and compressing to form a tablet, and
e. coating the tablet with one or more layers of aqueous dispersion comprising one or more film forming agents.

11. The process of claim 1 comprising the steps of:
a. blending amorphous cefditoren pivoxil and one or more pharmaceutically acceptable excipients to form a blend,
b. directly compressing the blend formed in step (a) into a tablet, and
c. coating the tablet with one or more layers of aqueous dispersion comprising one or more film forming agents.

12. The process of claim 1 comprising the steps of:
a. blending amorphous cefditoren pivoxil, a surfactant, and one or more pharmaceutically acceptable excipients to form a blend,
b. directly compressing the blend formed in step (a) into a tablet and
c. coating the tablets with one or more layers of aqueous dispersion of film forming agents.

13. The process of claim 1, wherein the solid dosage form further comprises one or more additional antibacterial agents.

14. A pharmaceutical composition comprising a solid dosage form, which comprises amorphous cefditoren pivoxil, optionally one or more pharmaceutically acceptable excipients and optionally one or more surfactants, wherein the solid dosage form is prepared by a dry process and is coated with one or more layers of aqueous dispersion comprising one or more film forming agents.

15. The pharmaceutical composition of claim 14, wherein the dry process is selected from dry granulation or direct compression.

16. The pharmaceutical composition of claim 14, wherein the one or more film forming agents are selected from one or more of hydroxypropyl methylcellulose, hydroxypropyl cellulose, methyl cellulose, carboxymethylcellulose, hydroxymethyl cellulose, hydroxyethylcellulose, ethyl cellulose, hydroxypropyl methyl phthalate, cellulose acetate, cellulose acetate trimelliatate, cellulose acetate phthalate, waxes, copolymers of acrylic and methacrylic acid or mixtures thereof.

17. The pharmaceutical composition of claim 14, wherein the one or more surfactants constitutes about 1 to 20% (w/w) based on the total weight of the solid dosage form and comprises at least one of anionic, cationic, zwitterionic or nonionic surfactants.

18. The pharmaceutical composition of claim 14, wherein the one or more pharmaceutically acceptable excipients comprise at least one or more fillers, binders, disintegrants, lubricants, glidants, coloring agents, flavoring agents or mixtures thereof.

19. The pharmaceutical composition of claim 14, wherein the solid dosage form further comprises one or more additional antibacterial agents.

20. A method for treating a bacterial infection by administering to a person in need thereof a pharmaceutical composition comprising a solid dosage form, which comprises amorphous cefditoren pivoxil, optionally one or more pharmaceutically acceptable excipients and optionally one or more surfactants, wherein the solid dosage form is prepared by a dry process and is coated with one or more layers of aqueous dispersion comprising one or more film forming agents.

21. The method of claim 20 further comprising concurrently or sequentially administering one or more additional antibacterial agents.
